(19) United States
(12) Patent Application Publication
(10) Pub. No.: US 2017/0265837 A1
(43) Pub. Date: Sep. 21, 2017

(54) RADIATION IMAGING SYSTEM, CONTROL APPARATUS, INFORMATION PROCESSING METHOD, AND STORAGE MEDIUM

(71) Applicant: CANON KABUSHIKI KAISHA, Tokyo (JP)

(72) Inventors: Tatsuya Suzuki, Tokyo (JP); Yuichi Nishii, Kawasaki-shi (JP)

(21) Appl. No.: 15/452,860

(22) Filed: Mar. 8, 2017

(30) Foreign Application Priority Data
Mar. 17, 2016 (JP) 2016-054465

(51) Int. Cl. A61B 6/00

(52) U.S. Cl. CPC A61B 6/586; A61B 6/563; A61B 6/54

(57) ABSTRACT

A radiation imaging system includes a radiation imaging apparatus which detects radiation transmitted through an object and obtains a radiation image, and a control apparatus which controls the radiation imaging apparatus. The radiation imaging system comprises: an abnormality information obtaining unit configured to obtain abnormality information indicating an abnormality occurring in the radiation imaging apparatus; and an examination information obtaining unit configured to obtain, from examination information stored in the control apparatus, examination information corresponding to the abnormality information.

### BACKGROUND OF THE INVENTION

Field of the Invention

The present invention relates to a radiation imaging system, a control apparatus, an information processing method, and a storage medium.

Description of the Related Art

A radiation imaging apparatus has been proposed which uses, as an image recording medium of the radiation imaging apparatus, a flat panel type radiation detector in place of a film cassette. A flat panel type radiation imaging apparatus is often operated while moving. However, a shock or the like given at the time of operation or, for example, a factor such as a temperature, a radio wave, or the like in a use environment may influence the normal operation of the radiation imaging apparatus.

Japanese Patent Application No. 2005-177379 has disclosed an arrangement including, in order to prohibit radiation irradiation if there is an abnormality in a radiation detector, a control apparatus which controls a radiation irradiation timing and a self-diagnostic circuit configured to diagnose the operable/inoperable state in a radiation imaging apparatus.

However, the arrangement of Japanese Patent Application No. 2005-177379 cannot specify examination information containing information such as an imaging condition, an imaging location, the imaging portion of an object, or the like when an abnormality is sensed in the radiation imaging apparatus. Accordingly, a similar abnormality may reoccur if an abnormality is caused by an operational method, the imaging location, or the like when an imaging operator performs radiation imaging based on the examination information.

The present invention has been made in consideration of the above-described problem, and provides a radiation imaging technique capable of associating abnormality information obtained when the abnormality is sensed in the radiation imaging apparatus with the examination information obtained when the abnormality is sensed.

### SUMMARY OF THE INVENTION

According to one aspect of the present invention, there is provided a radiation imaging system that includes a radiation imaging apparatus which detects radiation transmitted through an object and obtains a radiation image, and a control apparatus which controls the radiation imaging apparatus, the system comprising: an abnormality information obtaining unit configured to obtain abnormality information indicating an abnormality occurring in the radiation imaging apparatus; and an examination information obtaining unit configured to obtain, from examination information stored in the control apparatus, examination information corresponding to the abnormality information.

According to another aspect of the present invention, there is provided a control apparatus that controls a radiation imaging apparatus which detects radiation transmitted through an object and obtains a radiation image, the apparatus comprising: an obtaining unit configured to obtain, from examination information stored in the control apparatus, examination information which corresponds to abnormality information indicating an abnormality occurring in the radiation imaging apparatus.

According to another aspect of the present invention, there is provided an information processing method in a radiation imaging apparatus which detects radiation transmitted through an object and obtains a radiation image, the method comprising: obtaining abnormality information indicating an abnormality occurring in the radiation imaging apparatus; and obtaining, from examination information stored in a storage unit, examination information corresponding to the abnormality information.

According to the present invention, it becomes possible to associate abnormality information obtained when an abnormality is sensed in a radiation imaging apparatus with examination information obtained when an abnormality is sensed.

According to the present invention, it also becomes possible to specify the examination information obtained when the abnormality is sensed by outputting the abnormality information and the examination information when the abnormality is sensed in association with each other.

It further becomes possible to specify a user of the radiation imaging apparatus that has caused the abnormality.

Further features of the present invention will become apparent from the following description of exemplary embodiments (with reference to the attached drawings).

### BRIEF DESCRIPTION OF THE DRAWINGS

FIG. 1 is a block diagram showing the arrangement of a radiation imaging system according to the first embodiment;

FIG. 2 is a block diagram showing the arrangement of a radiation imaging apparatus according to the first embodiment;

FIG. 3 is a block diagram showing the arrangement of a control unit of a control apparatus according to the first embodiment;

FIG. 4 is a flowchart for explaining the processing sequence of an information processing method according to the first embodiment;

FIG. 5 is a view showing an example of abnormality information and specific information;

FIG. 6 is a block diagram showing the arrangement of a radiation imaging apparatus according to the second embodiment;

FIG. 7 is a flowchart for explaining the processing sequence of an information processing method according to the second embodiment;

FIG. 8 is a view showing the arrangement of a radiation imaging system according to the third embodiment;

FIG. 9 is a block diagram showing the arrangement of a control unit of a control apparatus according to the third embodiment;

FIGS. 10A and 10B are flowcharts for explaining the processing sequence of an information processing method according to the third embodiment;

FIG. 11 is a view showing an example of abnormality information and specific information according to the fourth embodiment;

FIG. 12 is a block diagram showing the arrangement of a control unit of a control apparatus according to the fourth embodiment; and

FIG. 13 is a view showing the arrangement of a radiation imaging system according to the fourth embodiment.

### DESCRIPTION OF THE EMBODIMENTS

Exemplary embodiments of the present invention will be described below in detail with reference to the accompanying drawings. Note that the constituent elements described in the embodiments are merely examples. The technical scope of the present invention is determined by the scope of claims and is not limited by the following individual embodiments. In this specification, radiation is not limited to X-rays but may be, for example, electromagnetic waves, α-rays, β-rays, γ-rays, or the like.

The arrangement of a radiation imaging system according to each embodiment will be described below with reference to the accompanying drawings. The arrangement of the radiation imaging system may be referred to as a radiation imaging apparatus. In the following description, however, an explanation will be given as the radiation imaging system.

### First Embodiment

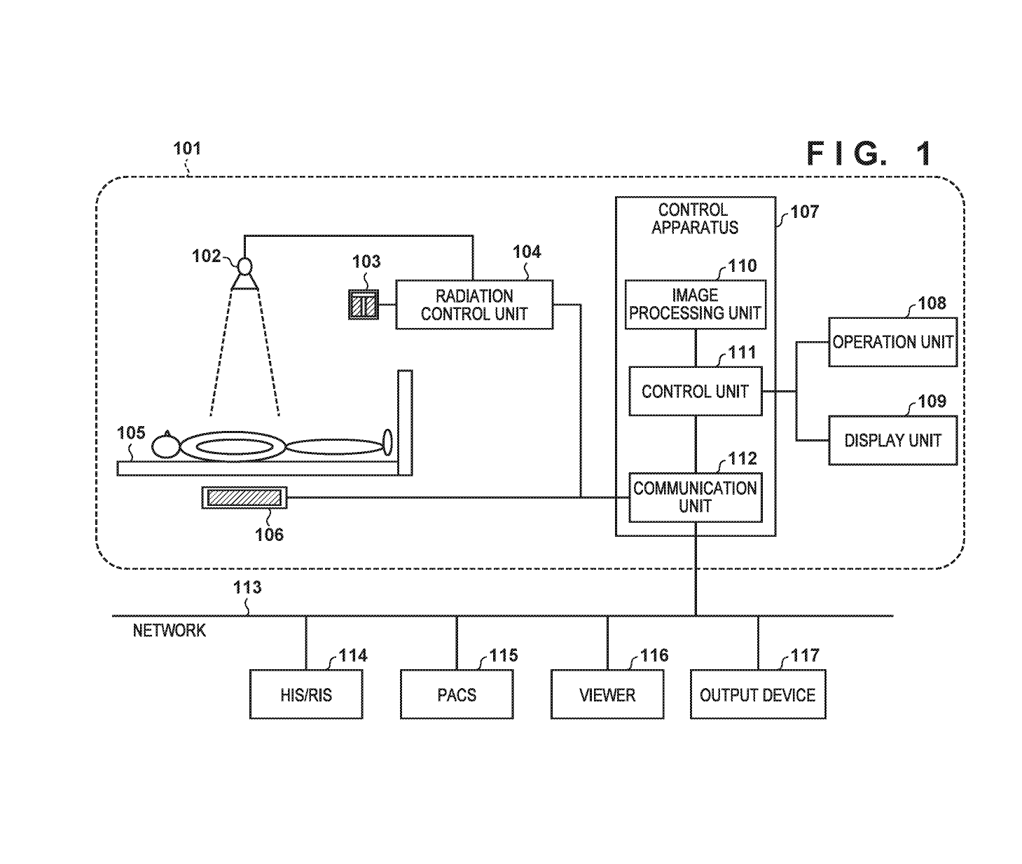

In this embodiment, the arrangement of a radiation imaging system will be described in which abnormality information obtained when an abnormality is sensed in a radiation imaging apparatus is associated with examination information obtained when an abnormality is sensed. FIG. 1 is a block diagram showing the arrangement of a radiation imaging system 101 according to the first embodiment. The radiation imaging system 101 includes a detection apparatus (radiation imaging apparatus 106) which detects radiation transmitted through an object and obtains a radiation image, and a control apparatus 107 which controls the detection apparatus (radiation imaging apparatus 106). The radiation imaging system 101 of this embodiment includes an abnormality information obtaining unit (data control unit 207) which obtains abnormality information indicating an abnormality occurring in the detection apparatus (radiation imaging apparatus 106) and an obtaining unit (examination information obtaining unit 301) which obtains examination information corresponding to the abnormality information from examination information of the object stored in the control apparatus 107. The radiation imaging system 101 also includes an addition unit (examination information addition unit 302) which generates specific information obtained by adding, to the abnormality information, the examination information obtained by the obtaining unit (examination information obtaining unit 301). The radiation imaging system 101 also includes, as constituent elements, a radiation generation apparatus 102, a radiation irradiation switch 103, a radiation control unit 104, a table 105, an operation unit 108, and a display unit 109.

The radiation generation apparatus 102 receives irradiation control information based on the examination information from the radiation control unit 104 and performs imaging preparation processing. The radiation generation apparatus 102 starts/stops radiation irradiation based on the control of the radiation control unit 104. Upon radiation irradiation, the radiation generation apparatus 102 transmits irradiation information on a tube voltage, a tube current, or the like to the radiation control unit 104. The radiation control unit 104 obtains the irradiation information on the tube voltage, the tube current, or the like transmitted from the radiation generation apparatus 102 and transmits the irradiation information obtained from the radiation generation apparatus 102 to the control apparatus 107. The control apparatus 107 stores the irradiation information obtained from the radiation control unit 104 together with the examination information in a storage unit.

The examination information contains, for example, information on a user of the control apparatus 107, information indicating an imaging condition, information indicating an imaging portion of the object, information indicating an imaging location, information on the examination schedule of the radiation imaging system, and information on the radiation imaging apparatus 106 used for an examination.

The radiation irradiation switch 103 transmits, to the radiation control unit 104, an irradiation start notification or an irradiation end notification. When the user presses the radiation irradiation switch 103, the radiation irradiation switch 103 transmits the irradiation start notification to the radiation control unit 104. On the other hand, when the user releases the radiation irradiation switch 103, the radiation irradiation switch 103 transmits the irradiation end notification to the radiation control unit 104. The radiation control unit 104 obtains the irradiation start notification or the irradiation end notification transmitted from the radiation irradiation switch 103 and based on the notification (the irradiation start notification or the irradiation end notification) from the radiation irradiation switch 103, controls radiation irradiation of the radiation generation apparatus 102.

The radiation control unit 104 is connected to the radiation generation apparatus 102, the radiation irradiation switch 103, and control apparatus 107 and transmits, to the control apparatus 107, the irradiation information transmitted from the radiation generation apparatus 102. Further, the radiation control unit 104 receives, from the control apparatus 107, information on an imaging condition (protocol) and the imaging portion of the object in the examination, and transmits, to the radiation generation apparatus 102, irradiation control information based on information on the received imaging condition and the imaging portion. Based on the irradiation control information received from the radiation control unit 104, the radiation generation apparatus 102 performs imaging preparation processing.

The table 105 is a gantry for laying an object H. The radiation imaging apparatus 106 detects radiation transmitted through the object H and converts it into radiation image data. The radiation imaging apparatus 106 is connected to the control apparatus 107 and transmits the radiation image data to the control apparatus 107. Upon sensing an abnormality in the radiation imaging apparatus 106, the radiation imaging apparatus 106 determines the date and time and an abnormality type at the time, and obtains abnormality information.

The radiation imaging apparatus 106 can communicate with the control apparatus 107 by wired communication using a cable or wireless communication. The radiation imaging apparatus 106 transmits the radiation image data or the abnormality information to the control apparatus 107 by wired communication or wireless communication. The radiation imaging apparatus 106 can also store the radiation image data or the abnormality information in the storage unit in the radiation imaging apparatus 106. For example, the radiation imaging apparatus 106 stores the radiation image data or the abnormality information in the internal storage unit when the radiation imaging apparatus 106 cannot communicate with the control apparatus 107 due to a communication status, and obtains the radiation image data or the abnormality information from the storage unit and transmits it to the control apparatus 107 when the radiation imaging apparatus 106 can communicate with the control apparatus 107.

The control apparatus 107 includes an image processing unit 110, a control unit 111, and a communication unit 112 and can control execution of radiation imaging, image processing, and an examination in cooperation with the radiation control unit 104 and the radiation imaging apparatus 106. The image processing unit 110 performs image processing of the radiation image data received by the communication unit 112. The image processing unit 110 can execute, for example, image processing such as tone processing or noise reduction processing for the radiation image data.

The control unit 111 generates, based on the examination information, control information for controlling execution of radiation imaging and the examination. The control unit 111 outputs, via the communication unit 112, the control information to the radiation control unit 104 and the radiation imaging apparatus 106 and based on the control information, controls execution of radiation imaging and the examination. In addition to performing control related to the examination and imaging, the control unit 111 can control saving of information on an examination to be conducted (suspended examination) and imaging information on a conducted examination (completed examination) or the radiation image data and readout of the radiation image data.

The communication unit 112 can communicate with the radiation control unit 104 and the radiation imaging apparatus 106 by wired communication using the cable or wireless communication, and can transmit/receive information to/from the radiation control unit 104 and the radiation imaging apparatus 106 via a communication interface (I/F). The communication unit 112 receives the irradiation information of the radiation generation apparatus 102 transmitted from the radiation control unit 104 and the radiation image data or the abnormality information transmitted from the radiation imaging apparatus 106, and transmits them to the control unit 111.

Upon accepting an operation input from the operation unit 108, the control apparatus 107 generates, based on input information, control information for controlling execution of radiation imaging and the examination. The operation unit 108 functions as an input interface which accepts an operation by the user. The operation unit 108 transmits the input information to the control apparatus 107 in accordance with the operation input. The operation unit 108 can be constituted of, for example, an input device such as a keyboard or mouse, or an input device in a form having a display unit such as a touch panel. Upon receiving a request from the control apparatus 107, the operation unit 108 switches the display of the input interface of the display unit such as the touch panel. The control apparatus 107 also functions as a display control apparatus which controls the display of the input interface of the display unit in the operation unit 108. For example, when the user designates, via the operation unit 108, the examination information in order to conduct the examination, the control apparatus 107 can control the display of the input interface so as to display detailed abnormality information (specific information) obtained by associating abnormality information sensed in a past examination with the examination information obtained when the abnormality is sensed. This allows the user to refer to and specify the examination information obtained when the abnormality is sensed, making it possible to reduce occurrence of an abnormality in radiation imaging in similar examination information.

Further, the control apparatus 107 performs display control for causing the display unit 109 to display the radiation image data obtained via the communication unit 112, a result of image processing by the image processing unit 110, and an examination result. The display unit 109 functions as an output interface which displays the user interface of control software in radiation imaging. The control apparatus 107 can also control the display of the display unit 109 so as to display the detailed abnormality information (specific information) obtained by associating the abnormality information sensed in the past examination with the examination information obtained when the abnormality is sensed. The user receiving informative display of the detailed abnormality information can refer to and specify the examination information obtained when the abnormality is sensed. This makes it possible to reduce occurrence of the abnormality in radiation imaging in the similar examination information.

Furthermore, the control apparatus 107 controls transmission/reception of information to/from an external apparatus via the communication unit 112 and a network 113. The control apparatus 107 of the radiation imaging system 101 is connected to, via the network 113, a HIS (Hospital Information System)/RIS (Radiology Information System) 114, PACS (Picture Archiving and Communication Systems) 115, a Viewer 116, and an output device 117. The HIS/RIS 114 is a hospital information system/radiology information management system which manages information such as patient information or examination request information in radiology. The PACS 115 is a server (picture archiving and communication system) mainly aiming at saving images. The Viewer 116 is connected to the PACS 115, and a high-definition monitor mainly executes inspection processing, detailed postprocessing, and diagnostic processing of an image captured in the radiation imaging system 101. The output device 117 can print out the radiation image data and the specific information.

[Arrangement of Radiation Imaging Apparatus 106]

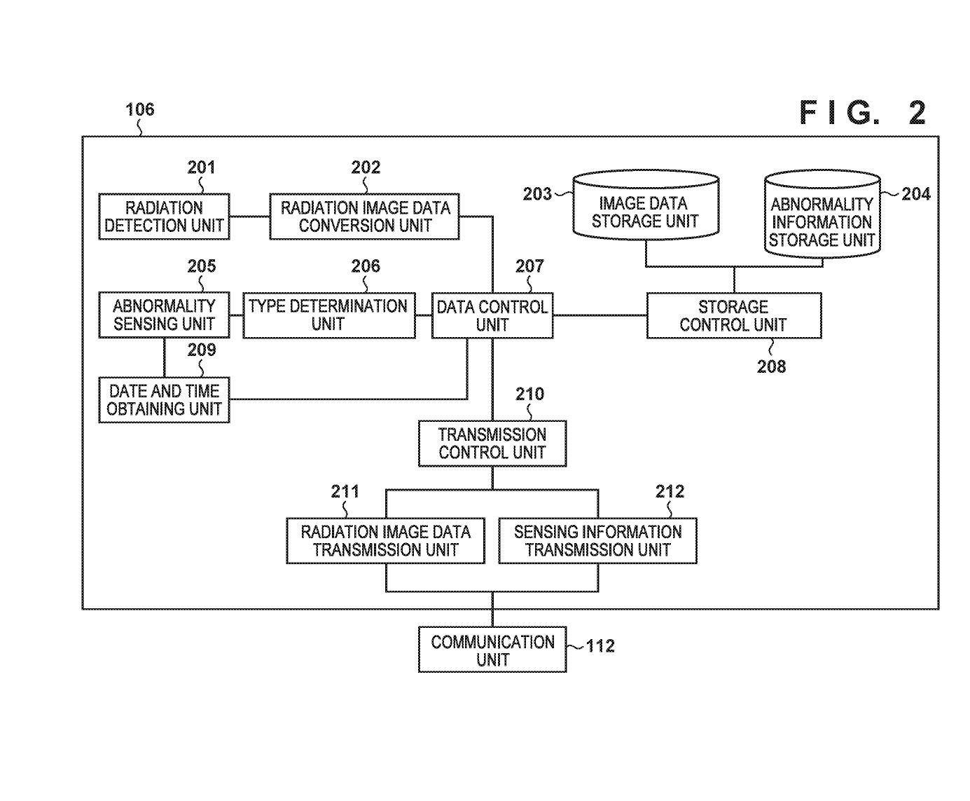

The detailed arrangement of the radiation imaging apparatus 106 according to this embodiment will now be described with reference to FIG. 2. The radiation imaging apparatus 106 includes a radiation detection unit 201, a radiation image data conversion unit 202, an image data storage unit 203, an abnormality information storage unit 204, an abnormality sensing unit 205, a type determination unit 206, the data control unit 207, a storage control unit 208, a date and time obtaining unit 209, a transmission control unit 210, a radiation image data transmission unit 211, and a sensing information transmission unit 212.

(Radiation Detection Unit 201)

The radiation detection unit 201 detects radiation emitted from the radiation generation apparatus 102. The radiation detection unit 201 detects the radiation transmitted through the object H as an image signal (charges). In the radiation detection unit 201, pixels which output signals corresponding to incident light are arranged in a two-dimensional region. A photoelectric conversion element of each pixel converts light converted by a phosphor into the image signal (charges) serving as an electrical signal, and a capacitor of each pixel performs accumulation.

When the radiation detection unit 201 detects radiation, the radiation image data conversion unit 202 converts an analog image signal output from the radiation detection unit 201 into digital image signal and transmits this as a radiation image (radiation image data) to the data control unit 207. The data control unit 207 receives the radiation image data transmitted from the radiation image data conversion unit 202, and transmits the received radiation image data to the transmission control unit 210 and the storage control unit 208.

(Abnormality Sensing Unit 205)

The abnormality sensing unit 205 senses the abnormality occurring in the radiation imaging apparatus 106. The abnormality sensing unit 205 includes a plurality of sensors in order to sense abnormalities, compares a measurement result of the plurality of sensors with a threshold serving as a reference for abnormality sensing, and determines abnormality sensing if the measurement result exceeds the threshold. The abnormality sensing unit 205 outputs information for identifying the measurement result of the plurality of sensors by adding it to an abnormality sensing determination result. The abnormality sensing unit 205 includes, as sensors for sensing abnormalities, an acceleration sensor for sensing an abnormality in a shock and a temperature sensor for sensing an abnormality in a temperature. The abnormality sensing unit 205 also includes, as sensors for sensing abnormalities, a communication time measurement unit which measures whether communication is completed within a predetermined time in order to sense an abnormality in communication and a charging amount sensing unit which senses an abnormality in insufficient battery charging. More specifically, the abnormality sensing unit 205 includes various sensing units configured to sense abnormalities, a communication time measurement unit which measures a communication time in order to determine whether communication is completed normally within a predetermined time, and the like. The abnormality sensing unit 205 compares a measurement result of the various sensing units and the communication time measurement unit with a threshold serving as a reference for abnormality sensing and determines abnormality sensing if the measurement result exceeds the threshold. The abnormality sensing unit 205 can sense the abnormalities, for example, the shock, the temperature abnormality, the communication abnormality, undercharging, and the like by using the various sensing units (for example, the acceleration sensor, the temperature sensor, a battery charging amount sensing unit), the communication time measurement unit (for example, a timer), and the like. Note that abnormality types are merely examples and limitation is not made to those examples. The abnormality sensing unit 205 outputs identification information (abnormality type identification information) for identifying the type of the various sensing units or the communication time measurement unit by adding it to an abnormality sensing result.

(Type Determination Unit 206)

The type determination unit 206 determines the abnormality type based on the output result of the abnormality sensing unit 205 added by the abnormality sensing unit 205. The type determination unit 206 determines the abnormality type based on information for identifying the measurement result. The type determination unit 206 can specify the type of abnormality sensing by referring to the identification information added to the abnormality sensing result. More specifically, the type determination unit 206 can specify, by referring to the identification information, whether the abnormality type is an abnormality to which a shock is given, the temperature abnormality, the communication abnormality, undercharging, or the like. When the abnormality sensing unit 205 senses the abnormality in the radiation imaging apparatus 106, the type determination unit 206 determines the kind of abnormality based on the identification information added by the abnormality sensing unit 205 and transmits a determination result of the kind of abnormality to the data control unit 207.

(Date and Time Obtaining Unit 209)

The date and time obtaining unit 209 obtains information indicating a sensing date and time of the abnormality occurred. The date and time obtaining unit 209 includes a timer which measures a time and calendar information. The date and time obtaining unit 209 is connected to the abnormality sensing unit 205 and receives a sensing result of the abnormality sensing unit 205 when the abnormality sensing unit 205 senses an abnormality. Upon receiving the sensing result of the abnormality sensing unit 205, the date and time obtaining unit 209 obtains the calendar information and time information of the timer, and transmits, to the data control unit 207, information indicating a date and time when the abnormality is sensed (abnormality sensing date and time).

(Data Control Unit 207)

The abnormality information obtaining unit (data control unit 207) obtains the abnormality information based on an abnormality type determination result and information indicating the abnormality sensing date and time. That is, the data control unit 207 obtains the abnormality information based on the abnormality type determination result input from the type determination unit 206 and the abnormality sensing date and time input from the date and time obtaining unit 209. The data control unit 207 can add identification information (information identification information) for identifying the abnormality information and the radiation image data to the abnormality information or the radiation image data.

The data control unit 207 transmits, to the transmission control unit 210 and the storage control unit 208, generated abnormality information or radiation image data obtained by performing radiation imaging in a normal state.

(Storage Control Unit 208)

The radiation imaging apparatus 106 includes the storage control unit 208 configured to control storage processing of the radiation image or storage processing of the abnormality information. The storage control unit 208 can switch storage units to use in accordance with the attribute of data to be stored. The storage control unit 208 uses the image data storage unit 203 as a storage area for the radiation image data and uses the abnormality information storage unit 204 as a storage area for the abnormality information. When information transmitted from the data control unit 207 is radiation image data, the storage control unit 208 stores the radiation image data in the image data storage unit 203. When information transmitted from the data control unit 207 is the abnormality information, the storage control unit 208 stores the abnormality information in the abnormality information storage unit 204.

As concrete processing, the storage control unit 208 identifies, based on the identification information (information identification information), whether the information transmitted from the data control unit 207 is the radiation image data or the abnormality information. Then, when the information received from the data control unit 207 is the abnormality information (the abnormality type determination result and the abnormality sensing date and time), the storage control unit 208 stores the abnormality information in the abnormality information storage unit 204. When the information received from the data control unit 207 is the radiation image data, the storage control unit 208 stores the radiation image data in the image data storage unit 203. The storage control unit 208 can also control, based on a data readout instruction from the data control unit 207, readout of the information stored in the image data storage unit 203 or the abnormality information storage unit 204.

(Transmission Control Unit 210)

Based on the identification information (information identification information) added to the abnormality information or the radiation image data, the transmission control unit 210 controls transmission processing of the information output from the radiation imaging apparatus 106. The transmission control unit 210 switches transmission units to use in accordance with the attribute (the abnormality information or the radiation image data) of data to be transmitted. The transmission control unit 210 uses the radiation image data transmission unit 211 in transmission processing of the radiation image data and uses the sensing information transmission unit 212 in transmission processing of the abnormality information.

When transmitting the radiation image data, the transmission control unit 210 controls the radiation image data transmission unit 211 to transmit the radiation image data from the radiation image data transmission unit 211 to the control apparatus 107. When transmitting the abnormality information, the transmission control unit 210 controls the sensing information transmission unit 212 to transmit the abnormality information from the sensing information transmission unit 212 to the control apparatus 107. The communication unit 112 of the control apparatus 107 receives the radiation image data or the abnormality information transmitted from the radiation imaging apparatus 106.

[Arrangement of Control Apparatus 107]

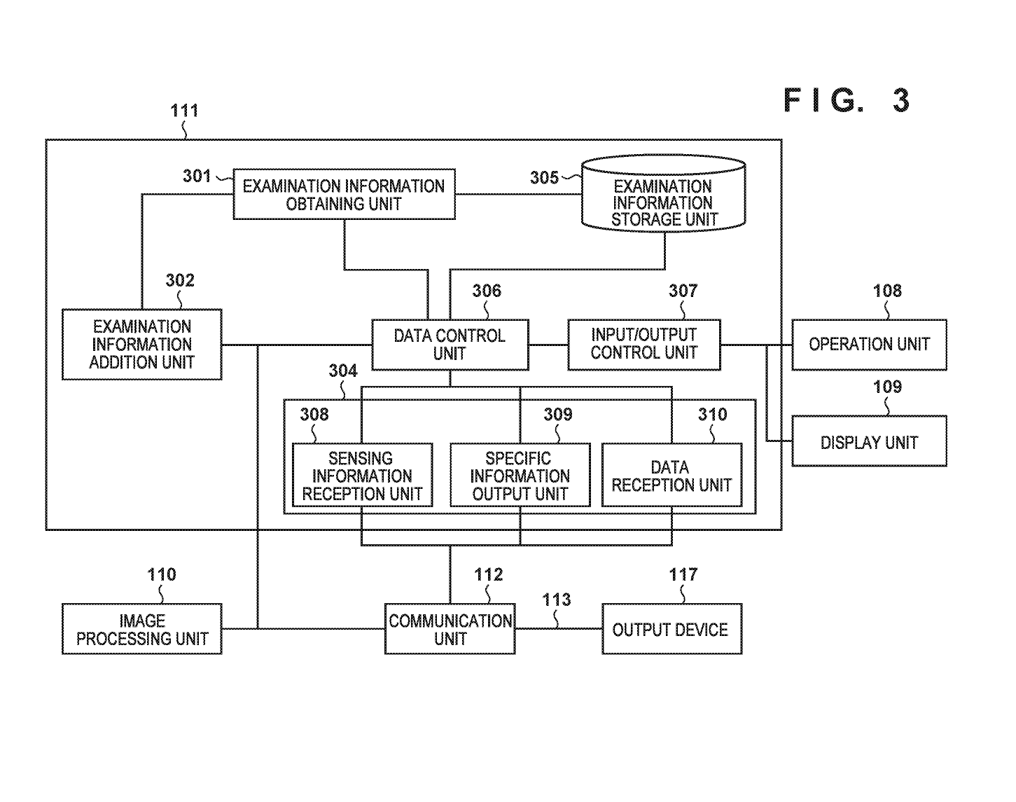

The detailed arrangement of the control unit 111 in the control apparatus 107 according to this embodiment will now be described with reference to FIG. 3. The control apparatus 107 controls the detection apparatus (radiation imaging apparatus 106) which detects the radiation transmitted through the object and obtains the radiation image. The control unit 111 of the control apparatus 107 includes the obtaining unit (examination information obtaining unit 301) which obtains, from the examination information of the object stored in the control apparatus, the examination information corresponding to the abnormality information indicating the abnormality occurring in the detection apparatus (radiation imaging apparatus 106). The control unit 111 of the control apparatus 107 also includes the addition unit (examination information addition unit 302) which generates the specific information obtained by adding, to the abnormality information, the examination information obtained by the obtaining unit (examination information obtaining unit 301). The control unit 111 also includes, as constituent elements, a transmission/reception control unit 304, an examination information storage unit 305, a data control unit 306, an input/output control unit 307, a sensing information reception unit 308, a specific information output unit 309, and a data reception unit 310.

(Data Control Unit 306)

The data control unit 306 controls an overall sequence for controlling registration and update of the patient information, the information on the examination to be conducted, and imaging technique information, screen display control, and execution of radiation imaging and the examination.

The examination information storage unit 305 can be formed as a database on the examination information. The data control unit 306 can, for example, register the examination information obtained from the HIS/RIS 114 in the examination information storage unit 305, or update or delete the registered examination information. For example, the examination information transmitted from the HIS/RIS 114 via the network 113 is received by the communication unit 112 and controlled by the transmission/reception control unit 304 of the control unit 111 to undergo reception processing by the data reception unit 310. Then, the data control unit 306 receives the examination information from the data reception unit 310, and performs registration and update processing of the obtained examination information.

(Input/Output Control Unit 307)

The input/output control unit 307 performs reception control of the input information from the operation unit 108. The input/output control unit 307 inputs the input information input from the operation unit 108 to the data control unit 306. The data control unit 306 controls execution of radiation imaging and the examination based on the input information. Further, the data control unit 306 outputs, to the input/output control unit 307, an execution result of radiation imaging and the examination. The input/output control unit 307 performs display control so as to display, on the display unit 109, the execution result of radiation imaging and the examination input from the data control unit 306.

(Examination Information Obtaining Unit 301)

The control apparatus 107 includes the storage unit (examination information storage unit 305) which stores a plurality of pieces of examination information. The obtaining unit (examination information obtaining unit 301) obtains the examination information corresponding to the abnormality information from the storage unit (examination information storage unit 305). Note that the obtaining unit (examination information obtaining unit 301) searches for examination information on an examination schedule corresponding to the abnormality sensing date and time of the abnormality information and obtains, from the storage unit (examination information storage unit 305), the examination information containing the examination schedule corresponding to the abnormality sensing date and time.

The abnormality information transmitted from the radiation imaging apparatus 106 is received by the communication unit 112 and controlled by the transmission/reception control unit 304 of the control unit 111 to undergo reception processing by the sensing information reception unit 308. Then, the data control unit 306 receives the abnormality information from the sensing information reception unit 308 and inputs the received abnormality information to the examination information obtaining unit 301. Based on the abnormality information, the data control unit 306 searches for the examination information registered in the examination information storage unit 305. Note that the abnormality information contains the information on the abnormality type determination result and the abnormality sensing date and time, and based on information, of the abnormality information, on the abnormality sensing date and time, the examination information obtaining unit 301 searches for the examination information stored in the examination information storage unit 305. The examination information contains information on the schedule of an examination to be conducted (expected examination date and time) in the radiation imaging system. The examination information obtaining unit 301 compares the abnormality sensing date and time of the abnormality information with the examination schedule (expected examination date and time) of the examination information stored in the examination information storage unit 305. Out of the examination information stored in the examination information storage unit 305, the examination information obtaining unit 301 searches for a latest examination schedule (expected examination date and time) which is closest to the abnormality sensing date and time of the abnormality information and specifies the examination information containing the examination schedule (expected examination date and time) corresponding to the abnormality sensing date and time. Then, the examination information obtaining unit 301 obtains the specified examination information from the examination information storage unit 305 and inputs the obtained examination information to the examination information addition unit 302. The data control unit 306 also inputs, to the examination information addition unit 302, the abnormality information input from the sensing information reception unit 308.

(Examination Information Addition Unit 302)

The examination information addition unit 302 adds the examination information obtained from the examination information obtaining unit 301 to the abnormality information obtained from the data control unit 306, and generates specific information based on the abnormality information and the examination information. Then, the examination information addition unit 302 inputs the generated specific information to the data control unit 306. The data control unit 306 can add, to the specific information, identification information (specific information identification information) for identifying the specific information, and the abnormality information and the radiation image data. The data control unit 306 inputs, to the transmission/reception control unit 304, the specific information obtained from the examination information addition unit 302.

(Transmission/Reception Control Unit 304)

The control apparatus 107 includes the transmission/reception control unit 304 which controls reception processing of the radiation image or the abnormality information and output processing of the specific information. The transmission/reception control unit 304 controls reception processing by the sensing information reception unit 308 and the data reception unit 310, and output processing by the specific information output unit 309. When receiving the information from the communication unit 112, the transmission/reception control unit 304 identifies, based on the identification information (information identification information) for identifying the abnormality information and the radiation image date, whether the received information is the abnormality information or the radiation image data. When receiving the abnormality information, the transmission/reception control unit 304 controls the sensing information reception unit 308 to perform reception processing of the abnormality information. The data control unit 306 receives the abnormality information from the sensing information reception unit 308 and inputs the obtained abnormality information to the examination information obtaining unit 301.

When receiving the radiation image data, the transmission/reception control unit 304 controls the data reception unit 310 to perform reception processing of the radiation image data. The data control unit 306 receives the radiation image data from the data reception unit 310 and inputs the obtained radiation image data to the image processing unit 110. The image processing unit 110 performs image processing of the radiation image data obtained from the data control unit 306. The image processing unit 110 can execute, for example, image processing such as tone processing or noise reduction processing for the radiation image data.

The control apparatus 107 includes an output unit which outputs the specific information based on the abnormality information and the examination information. Note that each of the transmission/reception control unit 304 and the specific information output unit 309 functions as the output unit. When receiving the specific information from the data control unit 306, the transmission/reception control unit 304 controls the specific information output unit 309 to output, from the specific information output unit 309, the specific information obtained from the data control unit 306. The specific information output from the specific information output unit 309 is output via the communication unit 112 and the network 113. The output specific information is output from the external output device 117 such as a printer. The input/output control unit 307 performs display control so as to display the specific information on the display unit 109. The user can specify the contents of the specific information by, for example, referring to the display on the display unit 109 and the output result of the output device 117. This makes it possible reduce occurrence of the abnormality in radiation imaging in the similar examination information.

(Specific Sequence of Examination Information)

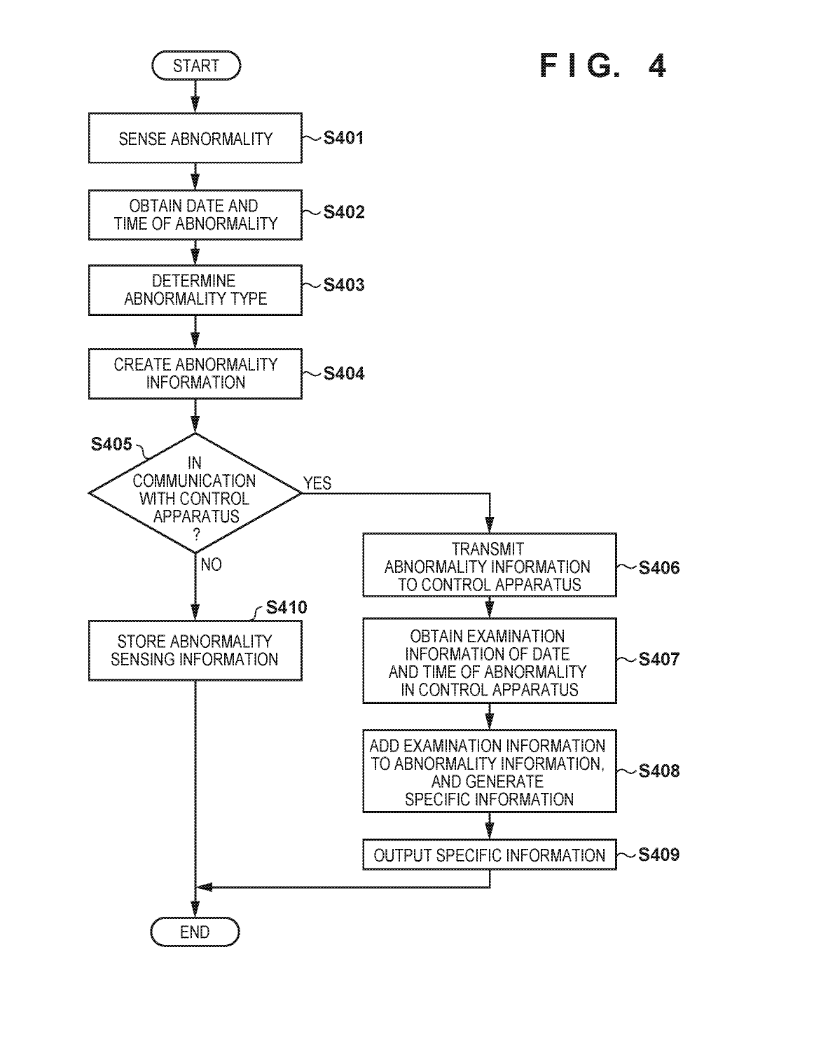

The processing sequence of an information processing method in the radiation imaging system according to this embodiment will now be described with reference to a flowchart of FIG. 4. First, in step S401, the abnormality sensing unit 205 of the radiation imaging apparatus 106 senses the abnormality in the radiation imaging apparatus 106. The abnormality sensing unit 205 compares the measurement result of the various sensors or the timer with the threshold serving as the reference for abnormality sensing and determines abnormality sensing if the measurement result exceeds the threshold. The abnormality sensing unit 205 adds, to the abnormality sensing result, the identification information (abnormality type identification information) for identifying the type of sensor or timer and outputs it to the data control unit 207.

In step S402, the date and time obtaining unit 209 of the radiation imaging apparatus 106 obtains the information indicating the date and time when the abnormality is sensed (abnormality sensing date and time). The date and time obtaining unit 209 is connected to the abnormality sensing unit 205 and receives the sensing result of the abnormality sensing unit 205 when the abnormality sensing unit 205 senses the abnormality. Upon receiving the sensing result of the abnormality sensing unit 205, the date and time obtaining unit 209 obtains the calendar information and the time information of the timer, and transmits, to the data control unit 207, the information indicating the date and time when the abnormality is sensed (abnormality sensing date and time).

Then, in step S403, the type determination unit 206 of the radiation imaging apparatus 106 determines the abnormality type. The abnormality sensing unit 205 can sense the abnormalities, for example, the shock, the temperature abnormality, the communication abnormality, undercharging, and the like by using the various sensors, the timer, and the like.

The type determination unit 206 includes, for example, the acceleration sensor as the arrangement for determining the abnormality type. If the acceleration sensor senses acceleration that exceeds predetermined threshold acceleration, the type determination unit 206 determines that an abnormal shock is given to the radiation imaging apparatus 106. The type determination unit 206 also includes, for example, the temperature sensor as the arrangement for determining the abnormality type. If the temperature sensor senses a temperature that exceeds a predetermined threshold temperature, the type determination unit 206 determines that the temperature of the radiation imaging apparatus 106 is abnormal.

The type determination unit 206 further includes, as the arrangement for determining the abnormality type, the communication time measurement unit which measures, for example, a time between the start and completion of communication with the control apparatus 107. If the measured communication time is longer than a reference time (threshold time), the type determination unit 206 determines communication abnormality. The type determination unit 206 also includes, as the arrangement for determining the abnormality type, the battery charging amount sensing unit which senses the charging amount of a battery (not shown) of the radiation imaging apparatus 106. For example, if the battery charging amount sensing unit senses a battery charging amount smaller than a predetermined reference battery remaining amount (threshold remaining amount), the type determination unit 206 can determine that the battery of the radiation imaging apparatus 106 is undercharged. Note that as the arrangement for determining the abnormality type, the arrangement of each of the acceleration sensor, the temperature sensor, the communication time measurement unit, and the battery charging amount sensing unit is merely an example, and is not limited to the above-described arrangement as long as it can implement the same function.

Then, in step S404, the data control unit 207 generates the abnormality information based on the abnormality type determination result input from the type determination unit 206 and the abnormality sensing date and time input from the date and time obtaining unit 209.

In step S405, if the radiation imaging apparatus 106 is in communication with the control apparatus 107 (Yes in step S405), the process advances to step S406. In step S406, when transmitting the abnormality information, the transmission control unit 210 controls the sensing information transmission unit 212 to transmit the abnormality information from the sensing information transmission unit 212 to the control apparatus 107.

In step S407, when receiving the information via the communication unit 112 of the control apparatus 107, the transmission/reception control unit 304 identifies, based on the identification information (information identification information) for identifying the abnormality information and the radiation image data, whether the input information is the abnormality information or the radiation image data. When receiving the abnormality information, the transmission/reception control unit 304 controls the sensing information reception unit 308 to perform reception processing of the abnormality information. The abnormality information is input, via the data control unit 306, from the sensing information reception unit 308 to the examination information obtaining unit 301.

The examination information obtaining unit 301 compares the abnormality sensing date and time of the abnormality information with the examination schedule (expected examination date and time) of the examination information stored in the examination information storage unit 305. Out of the examination information stored in the examination information storage unit 305, the examination information obtaining unit 301 searches for the examination schedule (expected examination date and time) closest to the abnormality sensing date and time of the abnormality information and specifies the examination information containing the examination schedule (expected examination date and time) corresponding to the abnormality sensing date and time. Then, the examination information obtaining unit 301 obtains the specified examination information from the examination information storage unit 305 and inputs the obtained examination information to the examination information addition unit 302.

In step S408, the examination information addition unit 302 adds the examination information obtained from the examination information obtaining unit 301 to the abnormality information obtained from the data control unit 306, and generates the specific information based on the abnormality information and the examination information. Then, the examination information addition unit 302 inputs the generated specific information to the data control unit 306. The data control unit 306 inputs the specific information obtained from the examination information addition unit 302 to the transmission/reception control unit 304.

Note that if the radiation imaging apparatus 106 is not in communication with the control apparatus 107 (No in step S405), the storage control unit 208 stores the abnormality information in the storage unit in the radiation imaging apparatus. When the radiation imaging apparatus 106 can communicate with the control apparatus 107, the storage control unit 208 can also transmit the abnormality information stored in the storage unit in the radiation imaging apparatus. In this case, the specific information is created with the examination information being “unknown”.

In step S409, when receiving the specific information from the data control unit 306, the transmission/reception control unit 304 controls the specific information output unit 309 to output, from the specific information output unit 309, the specific information obtained from the data control unit 306. The specific information output from the specific information output unit 309 is output, via the communication unit 112 and the network 113, to the output device 117.

On the other hand, if the radiation imaging apparatus 106 is not in communication with the control apparatus 107 in the determination in step S405 (No in step S405), the process advances to step S410. In step S410, the storage control unit 208 stores the abnormality information in the storage unit in the radiation imaging apparatus. That is, if the information received from the data control unit 207 is the abnormality information (the abnormality type determination result and the abnormality sensing date and time), the storage control unit 208 stores the abnormality information in the abnormality information storage unit 204.

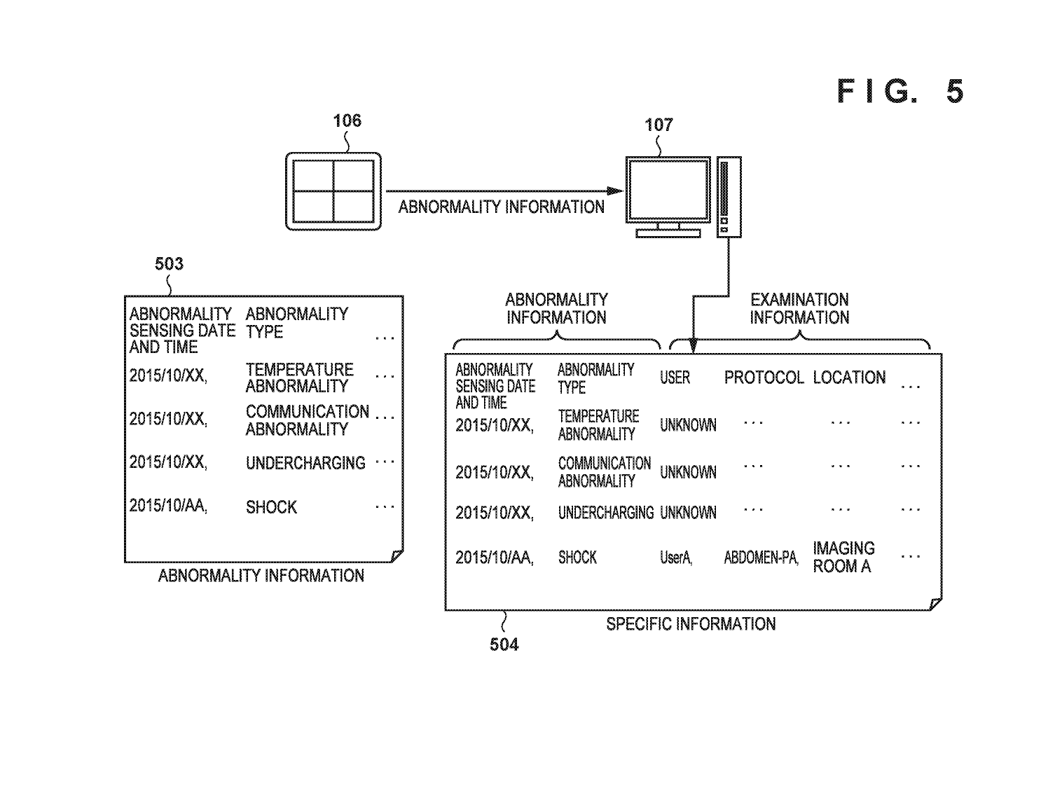

FIG. 5 is a view showing an example of the abnormality information stored in the abnormality information storage unit 204 of the radiation imaging apparatus 106 and the specific information output from the control apparatus 107. If the abnormality is sensed in the radiation imaging apparatus 106, the radiation imaging apparatus 106 transmits abnormality information 503 to the control apparatus 107. As shown in FIG. 5, the abnormality information 503 contains the abnormality sensing date and time and the abnormality types. The examination information obtaining unit 301 of the control apparatus 107 compares the abnormality sensing date and time of the abnormality information with the examination schedule (expected examination date and time) of the examination information stored in the examination information storage unit 305, searches for the latest examination schedule (expected examination date and time) which is closest to the abnormality sensing date and time of the abnormality information, and specifies the examination information containing the examination schedule (expected examination date and time) corresponding to the abnormality sensing date and time. Then, the examination information obtaining unit 301 obtains the specified examination information from the examination information storage unit 305. The examination information addition unit 302 of the control apparatus 107 adds, to the abnormality information, the examination information obtained by the examination information obtaining unit 301, and generates specific information 504 based on the abnormality information and the examination information.

In addition to the abnormality information of the abnormality sensing date and time and the abnormality type as in FIG. 5, the examination information of the user of the radiation imaging apparatus 106 that has caused the abnormality, imaging protocol, location, and the like is associated with the specific information 504. As shown in FIG. 5, a user (User A) is specified if the user inputs authentication information (a User ID and a password) to the radiation imaging apparatus 106 to use it. It is possible here to specify that the user (User A) used the radiation imaging apparatus 106 to cause a shock (abnormality) in the radiation imaging apparatus 106 on Oct. AA, 2015. “User A” is specified in the specific information 504. Note that if a user uses the radiation imaging apparatus 106 without inputting the authentication information (the User ID and the password), that is, if the user uses it as a guest, the user in the specific information 504 is not specified, and thus unknown. “Unknown” is specified in the specific information 504. The specific information 504 is not limited to a display in a text file format using the display unit 109 as in FIG. 5 but may be output by the output device 117 or the like. A system administrator of a hospital can specify the contents of the specific information by referring to the specific information. This makes it possible to reduce occurrence of the abnormality in radiation imaging in the similar examination information.

### Second Embodiment

In the first embodiment, the arrangement has been described in which the radiation imaging apparatus 106 transmits the abnormality information to the control apparatus 107 at the time of abnormality sensing, and the specific information obtained by adding the examination information to the abnormality information is generated by the control apparatus 107. In this embodiment, an arrangement will be described in which specific information generated by a control apparatus 107 is transmitted to a radiation imaging apparatus 106, and the specific information is stored in the radiation imaging apparatus 106.

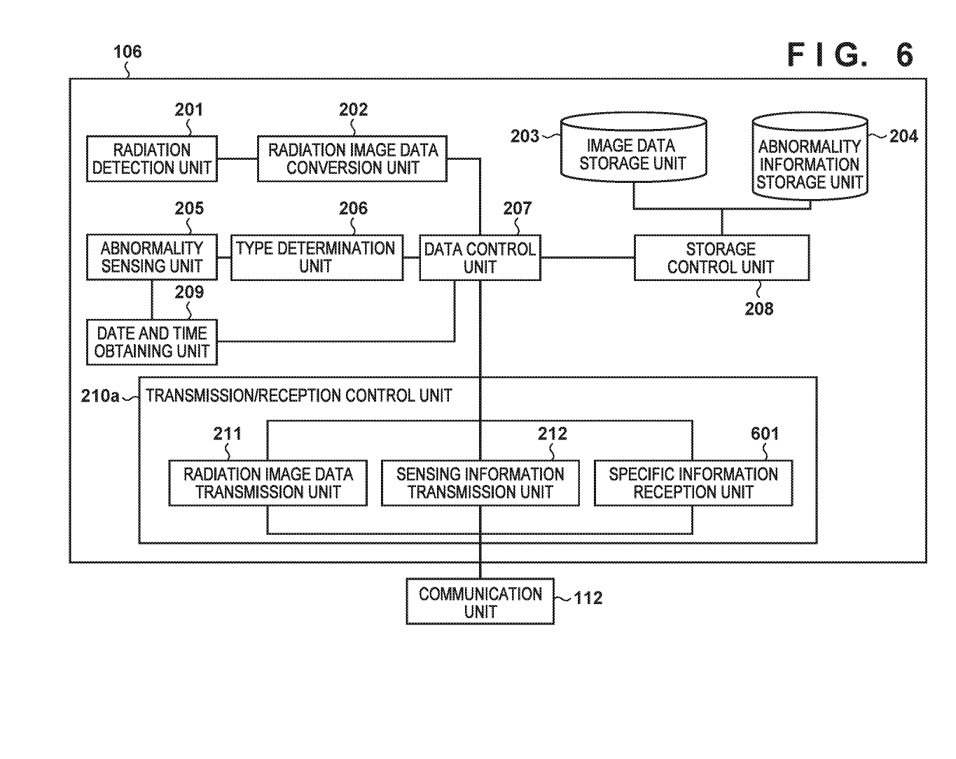

FIG. 6 is a block diagram showing the arrangement of the radiation imaging apparatus according to the second embodiment. The arrangement shown in FIG. 6 is almost the same as the arrangement of the radiation imaging apparatus 106 (FIG. 2) described in the first embodiment but is different in that a specific information reception unit 601 is added, and the transmission control unit 210 of FIG. 2 is changed to a transmission/reception control unit 210a. In a description below, processing of the specific information reception unit 601 and the transmission/reception control unit 210a serving as different components will be explained in order to avoid a repetition.

The radiation imaging apparatus 106 includes the transmission/reception control unit 210a configured to control transmission processing of a radiation image, transmission processing of abnormality information, or reception processing of the specific information output from the control apparatus 107. The transmission/reception control unit 210a controls reception processing of the specific information output from the control apparatus 107. A storage control unit 208 causes a storage unit (abnormality information storage unit 204) which stores the abnormality information to store the specific information.

When the specific information is transmitted from a communication unit 112 of the control apparatus 107, the transmission/reception control unit 210a of the radiation imaging apparatus 106 controls the specific information reception unit 601 to perform reception processing of the specific information. The specific information reception unit 601 receives the specific information transmitted from the communication unit 112 and inputs the received specific information to a data control unit 207. The data control unit 207 inputs, to the storage control unit 208, the specific information obtained from the specific information reception unit 601. A data control unit 306 of the control apparatus 107 adds identification information (specific information identification information) to the specific information. With this identification information, it is possible to identify the abnormality information and radiation image data, and the specific information. If the information obtained from the data control unit 207 is the specific information, the storage control unit 208 stores the specific information in the abnormality information storage unit 204. While the abnormality information storage unit 204 stores the abnormality information and the specific information, identification information (information identification information) for identifying the abnormality information and the radiation image data is added to the abnormality information. Identification information (specific information identification information) for identifying the abnormality information and the radiation image data, and the specific information is added to the specific information. Therefore, the abnormality information and the specific information can be distinguished from each other based on different types of identification information in the abnormality information storage unit 204.

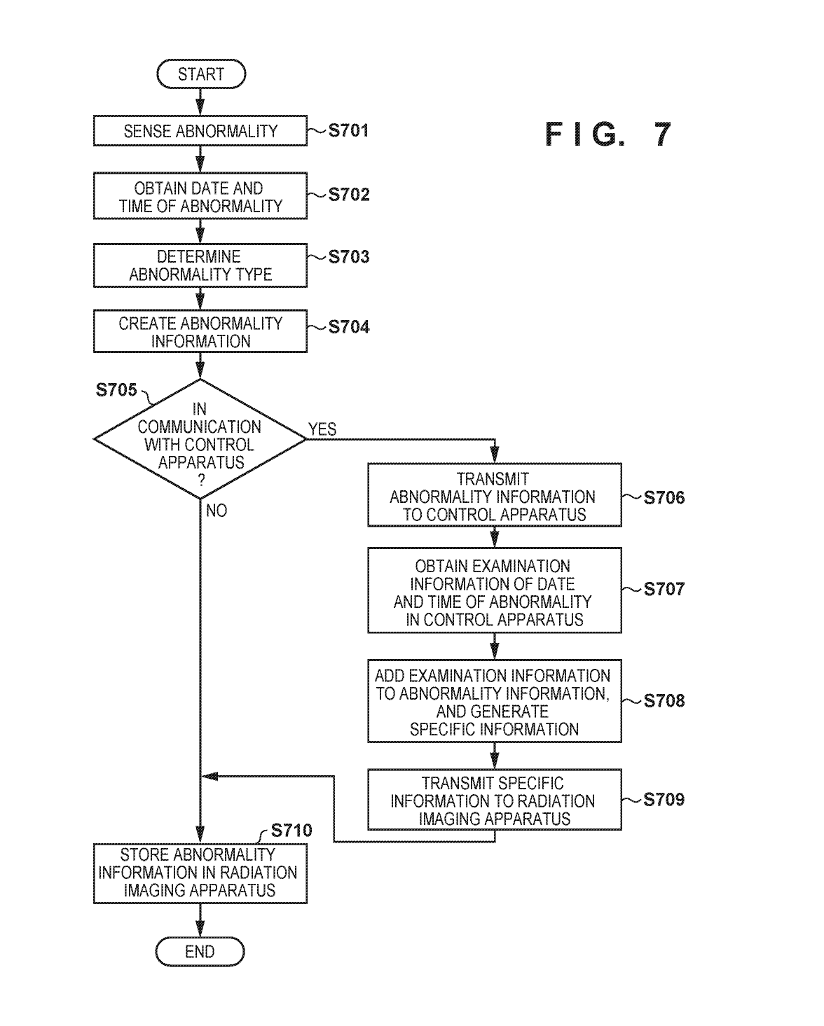

The processing sequence of an information processing method of a radiation imaging system according to the second embodiment will now be described with reference to a flowchart of FIG. 7.

First, in step S701, an abnormality sensing unit 205 of the radiation imaging apparatus 106 senses an abnormality in the radiation imaging apparatus 106. The abnormality sensing unit 205 compares a measurement result of various sensors and a timer with a threshold serving as a reference for abnormality sensing and determines abnormality sensing if the measurement result exceeds the threshold.

When the abnormality is sensed in the radiation imaging apparatus 106, in step S702, a date and time obtaining unit 209 of the radiation imaging apparatus 106 obtains information indicating a date and time when the abnormality is sensed (abnormality sensing date and time).

Then, in step S703, a type determination unit 206 of the radiation imaging apparatus 106 determines the abnormality type. The abnormality sensing unit 205 can sense abnormalities, for example, a shock, a temperature abnormality, a communication abnormality, undercharging, and the like by using the various sensors, a timer, and the like.

In step S704, the data control unit 207 generates abnormality information based on an abnormality type determination result input from the type determination unit 206 and the abnormality sensing date and time input from the date and time obtaining unit 209.

In step S705, if the radiation imaging apparatus 106 is in communication with the control apparatus 107 (Yes in step S705), the process advances to step S706. In step S706, when transmitting the abnormality information, the transmission/reception control unit 210a controls a sensing information transmission unit 212 to transmit the abnormality information from the sensing information transmission unit 212 to the control apparatus 107.

In step S707, when receiving the information via the communication unit 112 of the control apparatus 107, a transmission/reception control unit 304 identifies, based on the identification information (information identification information) for identifying the abnormality information and the radiation image data, whether the input information is the abnormality information or the radiation image data.

When receiving the abnormality information, the transmission/reception control unit 304 controls a sensing information reception unit 308 to perform reception processing of the abnormality information. The abnormality information is input, via the data control unit 306, from the sensing information reception unit 308 to an examination information obtaining unit 301.

The examination information obtaining unit 301 compares the abnormality sensing date and time of the abnormality information with an examination schedule (expected examination date and time) of examination information stored in an examination information storage unit 305. Out of the examination information stored in the examination information storage unit 305, the examination information obtaining unit 301 searches for an examination schedule (expected examination date and time) closest to the abnormality sensing date and time of the abnormality information, obtains, from the examination information storage unit 305, the examination information containing the examination schedule (expected examination date and time) corresponding to the abnormality sensing date and time, and inputs the obtained examination information to an examination information addition unit 302.

In step S708, the examination information addition unit 302 adds the examination information obtained from the examination information obtaining unit 301 to the abnormality information obtained from the data control unit 306, and generates specific information based on the abnormality information and the examination information.

In step S709, when receiving the specific information from the data control unit 306, the transmission/reception control unit 304 controls a specific information output unit 309 to transmit the specific information from the specific information output unit 309 to the radiation imaging apparatus 106.

Then, in step S710, when the specific information is transmitted from the communication unit 112 of the control apparatus 107, the transmission/reception control unit 210a of the radiation imaging apparatus 106 controls the specific information reception unit 601 to perform reception processing of the specific information. The specific information transmitted from the communication unit 112 is input, via the specific information reception unit 601 and the data control unit 207, to the storage control unit 208. If the information obtained from the data control unit 207 is the specific information, the storage control unit 208 stores the specific information in the abnormality information storage unit 204.

Note that if the radiation imaging apparatus 106 is not in communication with the control apparatus 107 in the determination in step S705 (No in step S705), it is impossible to obtain the examination information. Accordingly, the data control unit 207 generates specific information with the examination information being “unknown” and inputs it to the storage control unit 208. Then, the storage control unit 208 stores, in the abnormality information storage unit 204, the specific information obtained from the data control unit 207.

Based on a data readout instruction from the data control unit 207, the storage control unit 208 can control readout of information stored in an image data storage unit 203 or the abnormality information storage unit 204. For example, if the radiation imaging apparatus 106 is connected to a different control apparatus, the storage control unit 208 reads out, based on the data readout instruction from the data control unit 207, the specific information stored in the abnormality information storage unit 204. Then, the radiation imaging apparatus 106 transmits the specific information to the different control apparatus. This makes it possible to share the specific information obtained in the past between the different control apparatus and the radiation imaging apparatus 106 even if the different control apparatus and the radiation imaging apparatus 106 are newly connected to each other.

### Third Embodiment

In the second embodiment, the arrangement has been described in which the specific information obtained from the control apparatus 107 is stored in the storage unit (abnormality information storage unit 204) in the radiation imaging apparatus 106. In this embodiment, an arrangement will be described in which an external storage device stores specific information generated by a plurality of control apparatuses.

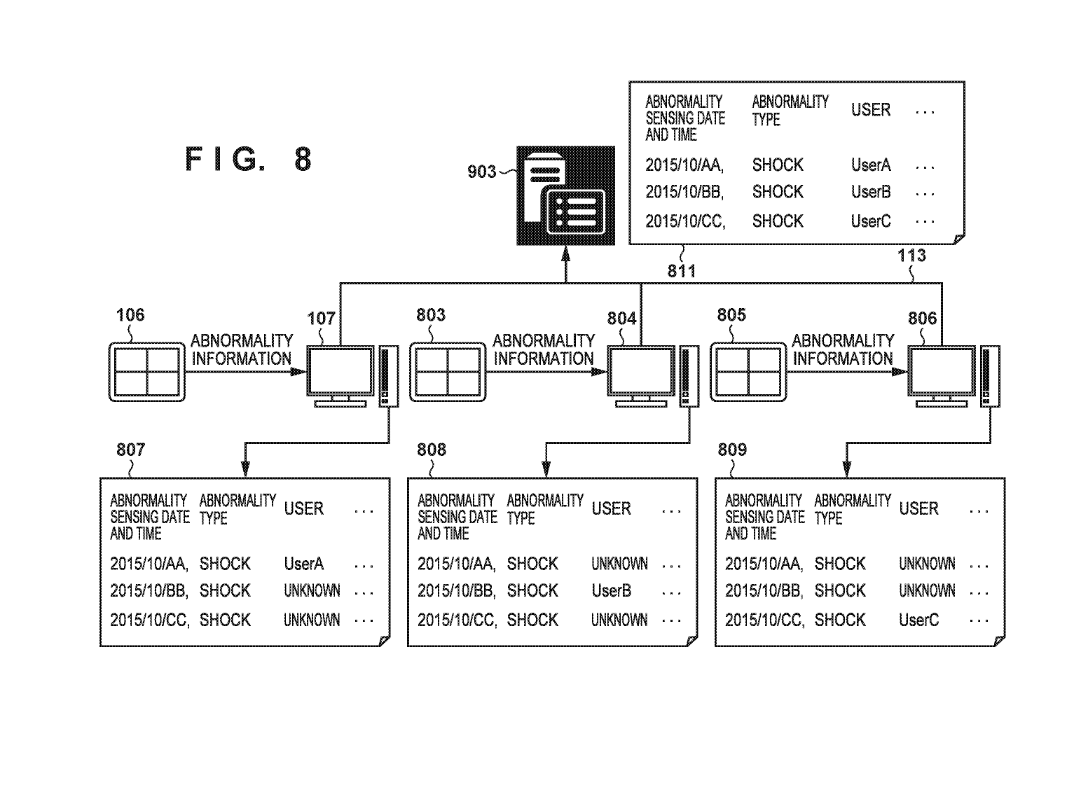

FIG. 8 is a view showing an example of the arrangement in which an external storage device 903 stores specific information generated by a plurality of control apparatuses 107, 804, and 806 in a radiation imaging system according to the third embodiment. The radiation imaging system of this embodiment includes the external storage device 903 which stores the specific information output from the control apparatuses. The external storage device 903 is connected to, via a network 113, the plurality of control apparatuses 107, 804, and 806 and stores a plurality of pieces of specific information obtained from the plurality of control apparatuses as integrated one piece of specific information.

In FIG. 8, radiation imaging apparatuses 106, 803, and 805 are, respectively, connected to the control apparatuses 107, 804, and 806. When abnormality information generated in the radiation imaging apparatuses 106, 803, and 805 is transmitted to the control apparatuses 107, 804, and 806, the control apparatuses 107, 804, and 806 generate pieces of specific information 807, 808, and 809 based on the abnormality information and outputs the generated pieces of specific information 807, 808, and 809.

The respective pieces of specific information output from the plurality of control apparatuses 107, 804, and 806 are stored, via the network 113, in the external storage device 903 which functions as a common database of the plurality of control apparatuses 107, 804, and 806. The external storage device 903 stores the plurality of pieces of specific information 807, 808, and 809 obtained from the plurality of control apparatuses 107, 804, and 806 as integrated one piece of specific information 811.

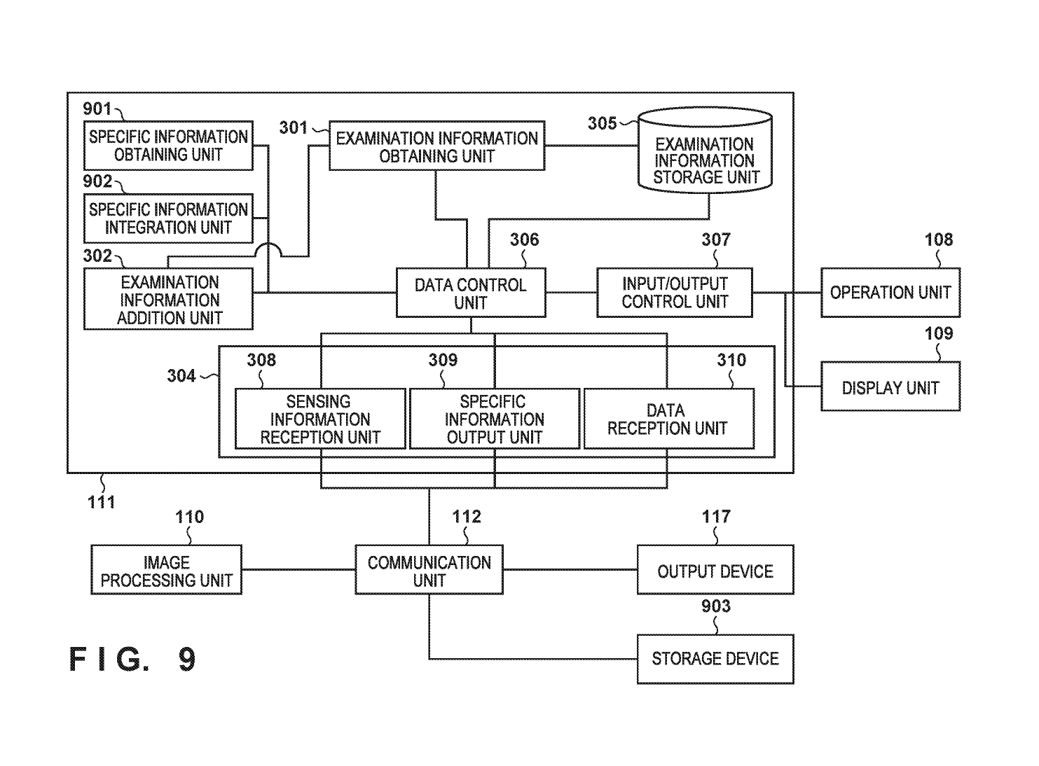

FIG. 9 is a block diagram for explaining the detailed arrangement of a control unit 111 in the control apparatus 107 according to the third embodiment. The arrangement shown in FIG. 9 is almost the same as the arrangement of the control apparatus 107 (FIG. 3) described in the second embodiment but is different in that a specific information obtaining unit 901, a specific information integration unit 902, and the external storage device 903 are added. In a description below, processing of the specific information obtaining unit 901, the specific information integration unit 902, and the external storage device 903 serving as different components will be explained in order to avoid a repetition. Note that the specific information obtaining unit 901 obtains the specific information from an external storage unit (external storage device 903). An integration unit (specific information integration unit 902) integrates specific information generated by an addition unit (examination information addition unit 302) and the specific information obtained from the external storage unit (external storage device 903) as one piece of specific information. Note that output units (a transmission/reception control unit 304 and a specific information output unit 309) of the control apparatus 107 output the integrated specific information to the external storage unit (external storage device 903). The output units (the transmission/reception control unit 304 and the specific information output unit 309) of the control apparatus 107 can also output the integrated specific information to an output device 117.

The external storage device 903 functions as the database of the control apparatus 107. The external storage device 903 stores the specific information. The pieces of specific information stored in the external storage device 903 contain the specific information generated by the control apparatus 107 and the specific information generated by the other control apparatuses.

The specific information obtaining unit 901 obtains, from the external storage device 903, the specific information via a data control unit 306, the transmission/reception control unit 304, and a communication unit 112.

The examination information addition unit 302 adds examination information obtained from an examination information obtaining unit 301 to abnormality information obtained from the data control unit 306, and generates specific information based on the abnormality information and the examination information.

Then, the specific information integration unit 902 obtains the specific information generated by the examination information addition unit 302 and the specific information that the specific information obtaining unit 901 obtains from the external storage device 903. Then, the specific information integration unit 902 integrates the specific information obtained from the external storage device 903 and the specific information generated by the examination information addition unit 302 as the one piece of specific information, and transmits it to the external storage device 903 via the data control unit 306 and the transmission/reception control unit 304.

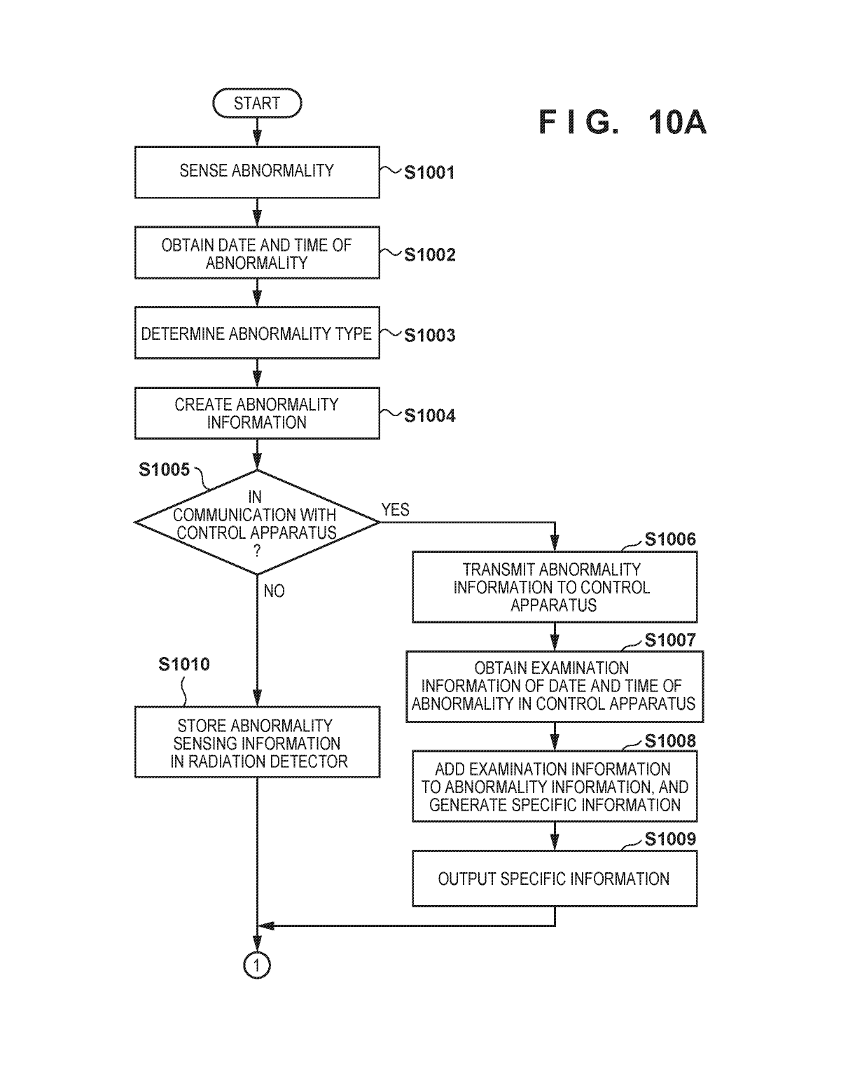

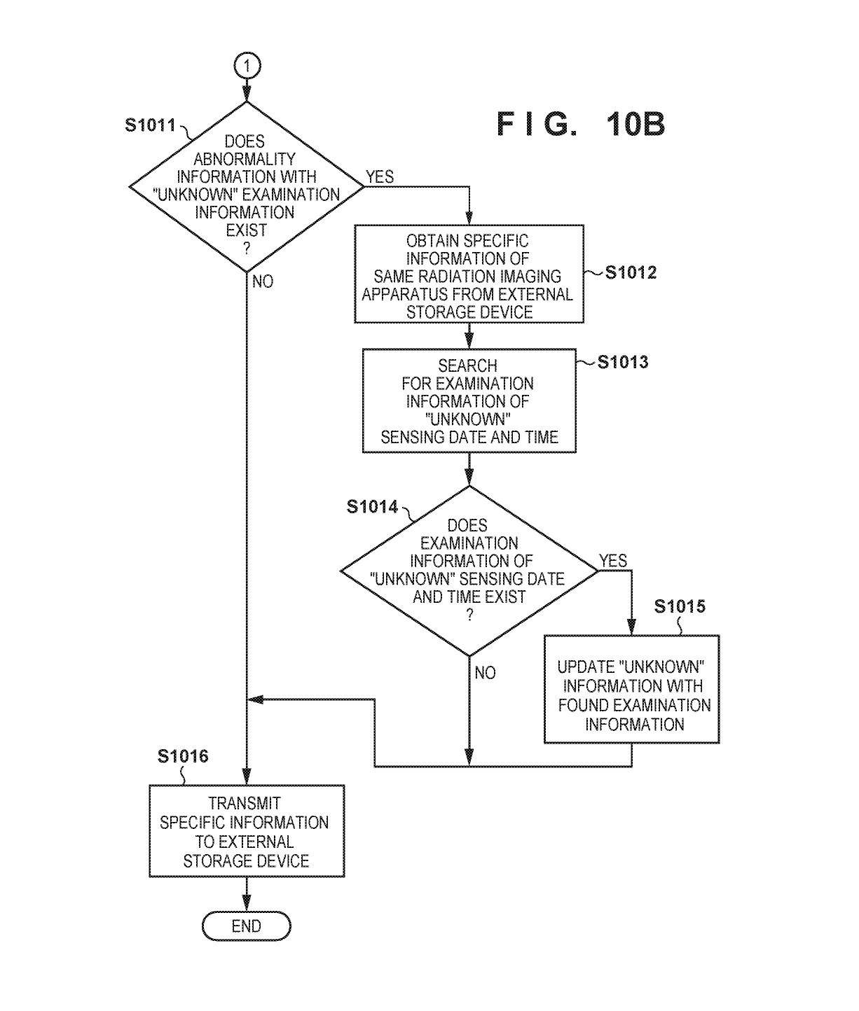

The processing sequence of an information processing method in the radiation imaging system according to the third embodiment will now be described with reference to flowcharts of FIGS. 10A and 10B. First, in step S1001, an abnormality sensing unit 205 of the radiation imaging apparatus 106 senses an abnormality in the radiation imaging apparatus 106. The abnormality sensing unit 205 compares a measurement result of various sensors or a timer with a threshold serving as a reference for abnormality sensing and determines abnormality sensing if the measurement result exceeds the threshold.

If the abnormality is sensed in the radiation imaging apparatus 106, in step S1002, a date and time obtaining unit 209 of the radiation imaging apparatus 106 obtains information indicating a date and time when the abnormality is sensed (abnormality sensing date and time).

Then, in step S1003, a type determination unit 206 of the radiation imaging apparatus 106 determines abnormality type. The abnormality sensing unit 205 can sense the abnormalities, for example, a shock, a temperature abnormality, a communication abnormality, undercharging, and the like by using the various sensors, the timer, and the like.

In step S1004, a data control unit 207 generates abnormality information based on an abnormality type determination result input from the type determination unit 206 and the abnormality sensing date and time input from the date and time obtaining unit 209.

In step S1005, if the radiation imaging apparatus 106 is in communication with the control apparatus 107 (Yes in step S1005), the process advances to step S1006. In step S1006, when transmitting the abnormality information, a transmission control unit 210 controls a sensing information transmission unit 212 to transmit the abnormality information from the sensing information transmission unit 212 to the control apparatus 107.

Then, in step S1007, when receiving the information via the communication unit 112 of the control apparatus 107, the transmission/reception control unit 304 identifies, based on identification information (information identification information) for identifying the abnormality information and radiation image data, whether the input information is the abnormality information or the radiation image data.

When receiving the abnormality information, the transmission/reception control unit 304 controls a sensing information reception unit 308 to perform reception processing of the abnormality information. The abnormality information is input, via the data control unit 306, from the sensing information reception unit 308 to the examination information obtaining unit 301.

The examination information obtaining unit 301 compares the abnormality sensing date and time of the abnormality information with an examination schedule (expected examination date and time) of examination information stored in an examination information storage unit 305. Out of the examination information stored in the examination information storage unit 305, the examination information obtaining unit 301 searches for an examination schedule (expected examination date and time) closest to the abnormality sensing date and time of the abnormality information, obtains, from the examination information storage unit 305, the examination information containing the examination schedule (expected examination date and time) corresponding to the abnormality sensing date and time, and inputs the obtained examination information to the examination information addition unit 302.

In step S1008, the examination information addition unit 302 adds the examination information obtained from the examination information obtaining unit 301 to the abnormality information obtained from the data control unit 306, and generates specific information based on the abnormality information and the examination information.

In step S1009, when receiving the specific information from the data control unit 306, the transmission/reception control unit 304 controls the specific information output unit 309 to output the specific information from the specific information output unit 309. The transmission/reception control unit 304 controls the specific information output unit 309 to transmit the specific information from the specific information output unit 309 to the radiation imaging apparatus 106.

On the other hand, if the radiation imaging apparatus 106 is not in communication with the control apparatus 107 in the determination in step S1005 (No in step S1005), the process advances to step S1010. In step S1010, a storage control unit 208 stores the abnormality information in a storage unit in the radiation imaging apparatus. That is, if the information received from the data control unit 207 is the abnormality information (the abnormality type determination result and the abnormality sensing date and time), the storage control unit 208 stores the abnormality information in an abnormality information storage unit 204. Note that if the radiation imaging apparatus 106 is not in communication with the control apparatus 107 in the determination in step S1005 (No in step S1005), it is impossible to obtain the examination information. Accordingly, the data control unit 207 generates specific information with the examination information being “unknown” and inputs it to the storage control unit 208. Then, the storage control unit 208 stores, in the abnormality information storage unit 204, the specific information obtained from the data control unit 207.

Then, in step S1011, if there is the abnormality information of the abnormality sensing date and time with the examination information being “unknown” in the specific information held by the examination information addition unit 302 of the control apparatus 107 (Yes in step S1011), the process advances to step S1012. Then, in step S1012, the specific information obtaining unit 901 of the control apparatus 107 obtains, from the external storage device 903, specific information of the same radiation imaging apparatus as the radiation imaging apparatus 106 connected to the control apparatus 107 and serving as a transmission source of the examination information.

Subsequently, in step S1013, the specific information obtaining unit 901 searches the obtained specific information for the examination information of the “unknown” abnormality sensing date and time. Then, in step S1014, if the examination information is found as a result of the search (Yes in step S1014), the process advances to step S1015. In step S1015, the examination information addition unit 302 updates the “unknown” examination information with the examination information found by the specific information obtaining unit 901. The examination information addition unit 302 generates specific information based on the found examination information and inputs the generated specific information to the specific information integration unit 902. The specific information integration unit 902 integrates the specific information obtained from the external storage device 903 and the specific information generated by the examination information addition unit 302 as one piece of specific information. Then, the specific information integration unit 902 inputs the integrated specific information to the data control unit 306. Then, in step S1016, the data control unit 306 inputs the specific information obtained from the specific information integration unit 902 to the transmission/reception control unit 304. The transmission/reception control unit 304 controls the specific information output unit 309 to transmit the specific information from the specific information output unit 309 to the external storage device 903.

On the other hand, in step S1014, if the examination information is not found as a result of the search (No in step S1014), the “unknown” examination information is not updated, and the process advances to step S1016. The examination information addition unit 302 inputs, to the data control unit 306, specific information with the examination information being “unknown”. Then, in step S1016, the data control unit 306 inputs the specific information obtained from the examination information addition unit 302 to the transmission/reception control unit 304. The transmission/reception control unit 304 controls the specific information output unit 309 to transmit the specific information from the specific information output unit 309 to the external storage device 903.

In step S1011, if there is not the abnormality information of the abnormality sensing date and time with the examination information being “unknown” in the specific information held by the examination information addition unit 302 of the control apparatus 107 (No in step S1011), in step S1016, the data control unit 306 inputs the specific information obtained from the examination information addition unit 302 to the transmission/reception control unit 304. The transmission/reception control unit 304 controls the specific information output unit 309 to transmit the specific information from the specific information output unit 309 to the external storage device 903.

According to this embodiment, the plurality of pieces of specific information specified by the plurality of control apparatuses are integrated as one piece of specific information and stored in the external storage device. With such an arrangement, it becomes possible to reduce a memory capacity in the radiation imaging apparatus 106. It becomes also possible to generate specific information by supplying the information with the examination information being “unknown” based on the specific information obtained from the external storage device if there is information with the examination information being “unknown” in the specific information held by the control apparatus 107 when, for example, not communicating with the radiation imaging apparatus 106.

### Fourth Embodiment

In the fourth embodiment, an arrangement will be described in which a user of a radiation imaging apparatus that has caused an abnormality is specified in a radiation imaging system.

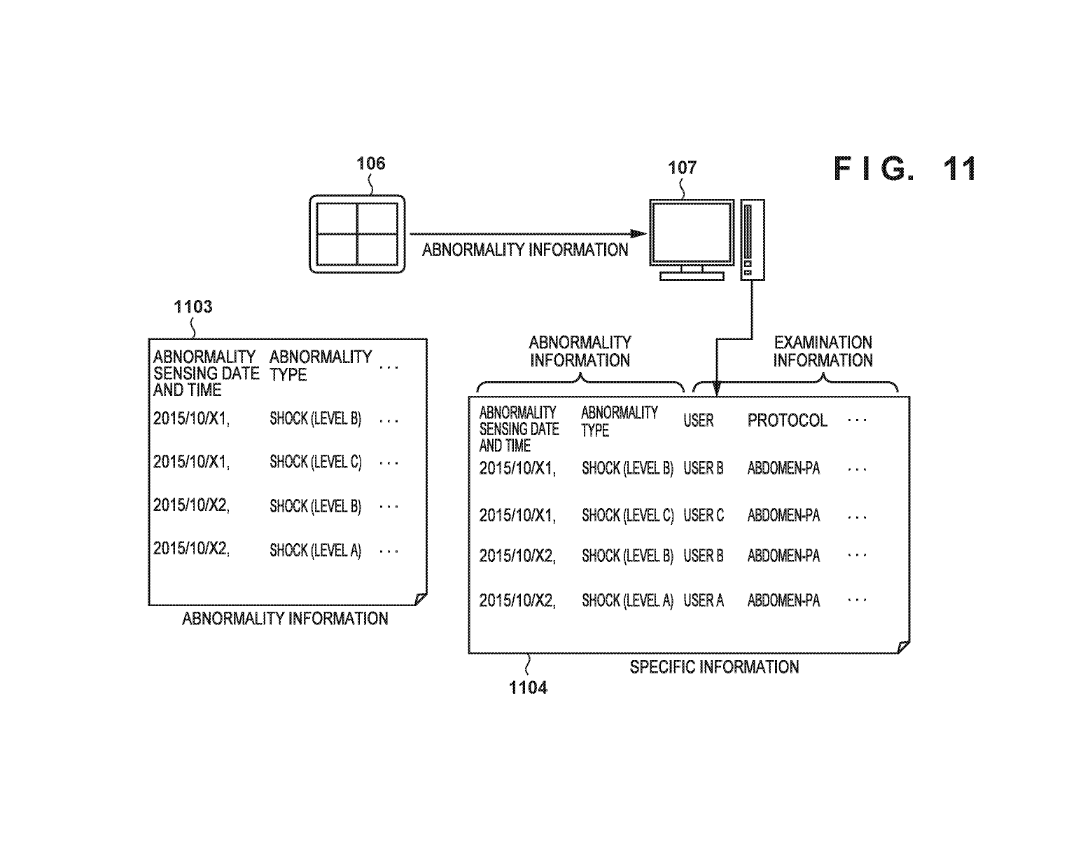

FIG. 11 is a view showing an example of abnormality information stored in an abnormality information storage unit 204 of a radiation imaging apparatus 106 and specific information output from a control apparatus 107. If an abnormality is sensed in the radiation imaging apparatus 106, the radiation imaging apparatus 106 transmits abnormality information 1103 to the control apparatus 107.

As shown in FIG. 11, the abnormality information 1103 contains an abnormality sensing date and time and an abnormality type. The abnormality type contains information indicating abnormality levels. For example, taking shocks as the abnormality type as an example, level A indicates the highest abnormality level (shock), level B indicates the medium abnormality level, and level C indicates the lowest abnormality level.

An examination information obtaining unit 301 (specifying unit) of the control apparatus 107 compares the abnormality sensing date and time of the abnormality information and an examination schedule (expected examination date and time) of examination information stored in an examination information storage unit 305, searches for a latest examination schedule (expected examination date and time) which is closest to the abnormality sensing date and time of the abnormality information, and specifies the examination information containing the examination schedule (expected examination date and time) corresponding to the abnormality sensing date and time.

An examination information addition unit 302 of the control apparatus 107 adds, to the abnormality information, the examination information obtained by the examination information obtaining unit 301, and generates specific information 1104 based on the abnormality information and the examination information.

Note that the examination information corresponding to the abnormality information 1103 contains information on the user of the radiation imaging apparatus 106 that has caused the abnormality. In this embodiment, when using the radiation imaging apparatus 106, the user needs to input authentication information (a User ID and a password) for specifying the user so as to prevent the user from being unknown. Based on the input authentication information, the user is allowed to use the radiation imaging apparatus 106. The examination information contains the authentication information as the user information. Based on the examination information corresponding to the abnormality information 1103, the examination information obtaining unit 301 (specifying unit) of the control apparatus 107 can specify the user of the radiation imaging apparatus 106 that has caused the abnormality.

The examination information obtaining unit 301 (specifying unit) can specify that, for example, User B used the radiation imaging apparatus 106 to cause a shock (abnormality) at level B in the radiation imaging apparatus 106 on Oct. X1, 2015. The examination information obtaining unit 301 (specifying unit) can also specify that, User C used the radiation imaging apparatus 106 to cause a shock (abnormality) at level C in the radiation imaging apparatus 106 on Oct. X1, 2015.

The examination information obtaining unit 301 (specifying unit) can specify, based on the authentication information input by the user, a user who has used the radiation imaging apparatus 106 and can also specify, based on a history of the input authentication information, a last user who has used the radiation imaging apparatus 106. For example, in the example shown in FIG. 11, the examination information obtaining unit 301 (specifying unit) can specify that user A last used the radiation imaging apparatus 106 to cause a shock (abnormality) at level A in the radiation imaging apparatus 106 on Oct. X2, 2015.

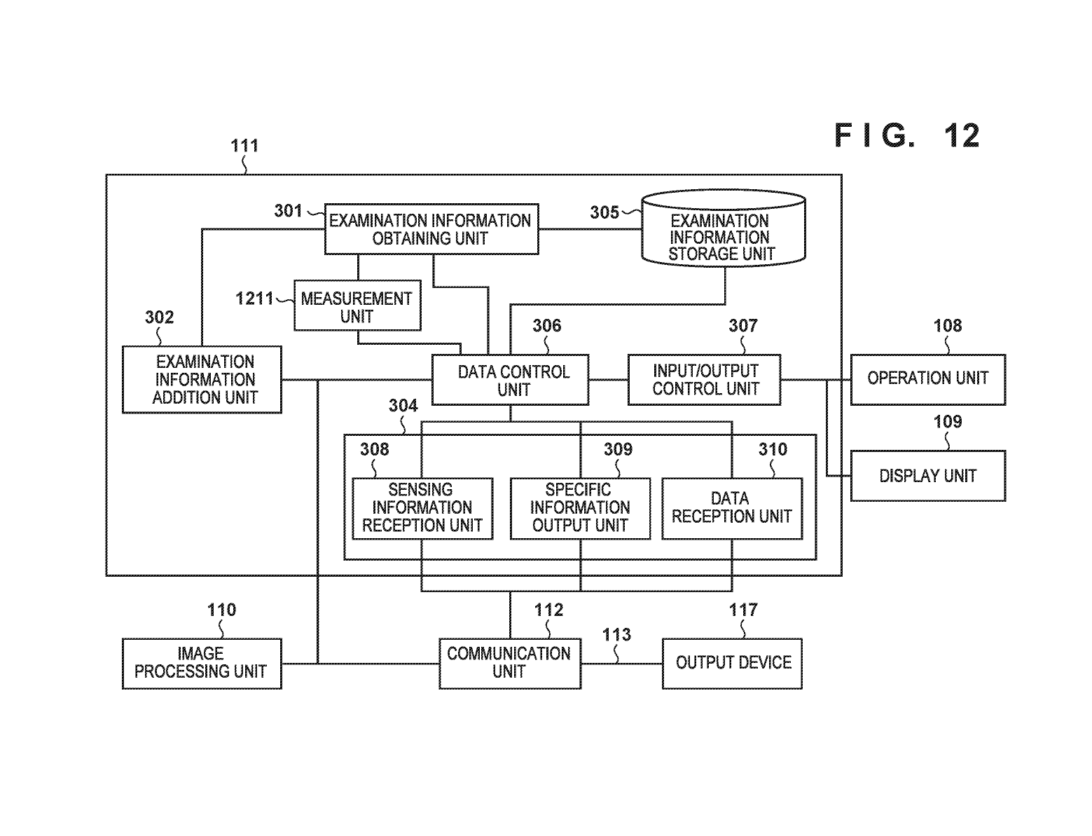

FIG. 12 is a block diagram for explaining the arrangement of a control unit 111 in the control apparatus 107 according to the fourth embodiment. In contrast to the arrangement of FIG. 3, a measurement unit 1211 is added to the control unit 111 of this embodiment. The measurement unit 1211 measures an abnormality sensing count of the user who has caused the abnormality in the radiation imaging apparatus 106. The examination information obtaining unit 301 (specifying unit) specifies, based on the user information (authentication information), the user who has caused the abnormality in the radiation imaging apparatus 106, and the information (authentication information) on the specified user is input to the communication unit 112. Upon receiving the user information (authentication information), the measurement unit 1211 measures the abnormality sensing count of the user who has caused the abnormality. For example, in the example of FIG. 11, User B causes the abnormality twice, and each of User A and User C causes the abnormality once. A measurement result of the abnormality sensing count by the measurement unit 1211 is output via a data control unit 306 and a specific information output unit 309.

Based on the measurement result of the abnormality sensing count by the measurement unit 1211, it is possible to specify the user (for example, User B in the example of FIG. 11) who causes an abnormality at a high frequency in the radiation imaging system. Based on the measurement result of the measurement unit 1211, a system administrator of a hospital can give guidance on, for example, an improvement in usage to the user who causes the abnormality at the high frequency.

The abnormality information 1103 contains the information indicating the abnormality levels. The examination information corresponding to the abnormality information contains the information on the user of the radiation imaging apparatus 106 that has caused the abnormality. Based on a comparison of the information indicating the abnormality levels, the examination information obtaining unit 301 (specifying unit) can specify the user of the radiation imaging apparatus 106 that has caused the largest abnormality out of the abnormalities occurred. For example, in the example of FIG. 11, the user (User A) of the radiation imaging apparatus 106 that has caused the largest abnormality (shock level A) is specified. The user of the radiation imaging apparatus 106 that has caused the largest abnormality out of the abnormalities having occurred is highly likely to do damage to the radiation imaging apparatus 106. Based on a specified result of the examination information obtaining unit 301 (specifying unit), the system administrator of the hospital can give the guidance on, for example, the improvement in usage to the user who has caused the largest abnormality.

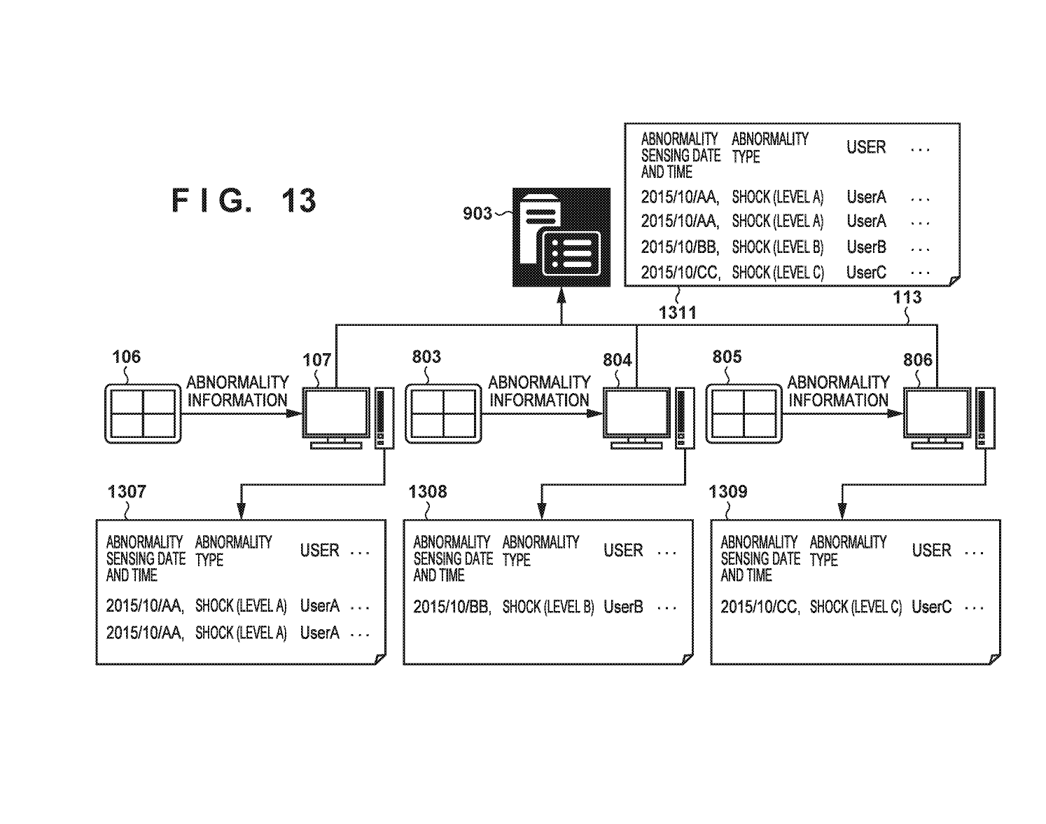

FIG. 13 is a view showing an example of the arrangement in which specific information generated by a plurality of control apparatuses 107, 804, and 806 is stored in an external storage device 903 in the radiation imaging system according to the fourth embodiment. The radiation imaging system of this embodiment includes the external storage device 903 which stores pieces of specific information 1307, 1308, and 1309 output from the plurality of control apparatuses 107, 804, and 806. The external storage device 903 is connected to the plurality of control apparatuses 107, 804, and 806 via a network 113 and stores the plurality of pieces of specific information 1307, 1308, and 1309 obtained from the plurality of control apparatuses as integrated one piece of specific information 1311.

In FIG. 13, the radiation imaging apparatus 106 and radiation imaging apparatuses 803 and 805 are, respectively, connected the control apparatuses 107, 804, and 806. When the abnormality information generated in the radiation imaging apparatuses 106, 803, and 805 is transmitted to the control apparatuses 107, 804, and 806, the control apparatuses 107, 804, and 806 generate the pieces of specific information 1307, 1308, and 1309 based on the abnormality information and outputs the generated pieces of specific information 1307, 1308, and 1309.

In the example shown in FIG. 13, User A used the radiation imaging apparatus 106 twice to cause the shock (abnormality) at level A in the radiation imaging apparatus 106 on Oct. AA, 2015. Further, User B used the radiation imaging apparatus 803 to cause the shock (abnormality) at level B in the radiation imaging apparatus 803 on Oct. BB, 2015. Furthermore, User C used the radiation imaging apparatus 805 to cause the shock (abnormality) at level C in the radiation imaging apparatus 805 on Oct. CC, 2015.

The external storage device 903 stores the specific information 1311 obtained by integrating the plurality of pieces of specific information 1307, 1308, and 1309 obtained from the plurality of control apparatuses 107, 804, and 806 into one and functions as a database of each control apparatus. In the radiation imaging system, each control apparatus can specify the level of the abnormality occurring in each radiation imaging apparatus, the user who has caused the abnormality, and the abnormality sensing count of each user by referring to the specific information 1311 stored in the external storage device 903 via the data control unit 306, a transmission/reception control unit 304, and a communication unit 112.

The radiation imaging system (specifying unit) can specify, based on the abnormality sensing count, the user (for example, User A in the example of FIG. 13) who causes an abnormality at a high frequency. The system administrator of the hospital can give the guidance on, for example, the improvement in usage to the user who causes the abnormality at the high frequency.

The radiation imaging system (specifying unit) can also specify, based on the information indicating the abnormality levels, the user of the radiation imaging apparatus that has caused the largest abnormality out of the abnormalities having occurred. For example, in the example of FIG. 13, User A of the radiation imaging apparatus that has caused the largest abnormality (shock level A) is specified. The user of the radiation imaging apparatus that has caused the largest abnormality out of the abnormalities having occurred is highly likely to do damage to the radiation imaging apparatus. The system administrator of the hospital can give the guidance on, for example, the improvement in usage to the user who has caused the largest abnormality.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2016-054465, filed Mar. 17, 2016, which is hereby incorporated by reference herein in its entirety.

What is claimed is:

1. A radiation imaging system that includes a radiation imaging apparatus which detects radiation transmitted through an object and obtains a radiation image, and a control apparatus which controls the radiation imaging apparatus, the system comprising:
an abnormality information obtaining unit configured to obtain abnormality information indicating an abnormality occurring in the radiation imaging apparatus; and
an examination information obtaining unit configured to obtain, from examination information stored in the control apparatus, examination information corresponding to the abnormality information.

2. The system according to claim 1, wherein the radiation imaging apparatus includes
an abnormality sensing unit configured to sense the abnormality occurring in the radiation imaging apparatus,
a type determination unit configured to determine a type of the abnormality based on an output result of the abnormality sensing unit, and
a date and time obtaining unit configured to obtain information indicating a sensing date and time of the abnormality having occurred, and
the abnormality information obtaining unit obtains the abnormality information based on a determination result of the type of the abnormality and the information indicating the sensing date and time of the abnormality.

3. The system according to claim 2, wherein the abnormality sensing unit includes a plurality of sensors in order to sense abnormalities, and
compares a measurement result of the plurality of sensors and a threshold serving as a reference for abnormality sensing, and determines abnormality sensing if the measurement result exceeds the threshold.

4. The system according to claim 3, wherein the abnormality sensing unit outputs information for identifying the measurement result of the plurality of sensors by adding the information to a determination result of the abnormality sensing.

5. The system according to claim 4, wherein the type determination unit determines the type of the abnormality based on the information for identifying the measurement result.

6. The system according to claim 1, further comprising an addition unit configured to generate specific information obtained by adding the obtained examination information to the abnormality information.

7. The system according to claim 6, wherein the control apparatus further includes a transmission/reception control unit configured to control reception processing of one of the radiation image and the abnormality information, and output processing of the specific information.

8. The system according to claim 7, further comprising an external storage unit configured to store the specific information output from the control apparatus.

9. The system according to claim 8, wherein the external storage unit is connected to a plurality of control apparatuses via a network and stores a plurality of pieces of specific information obtained from the plurality of control apparatuses as integrated one piece of specific information.

10. The system according to claim 8, wherein the control apparatus further includes
a specific information obtaining unit configured to obtain the specific information from the external storage unit, and
an integration unit configured to integrate the specific information generated by the addition unit and the specific information obtained from the external storage unit as one piece of specific information, and
an output unit of the control apparatus outputs the integrated specific information to the external storage unit.

11. The system according to claim 1, wherein the examination information contains information on a user of the control apparatus, information indicating an imaging condition, information indicating an imaging portion of the object, information indicating an imaging location, information on an examination schedule of the radiation imaging system, and information on the radiation imaging apparatus used in an examination.

12. The system according to claim 3, wherein the abnormality sensing unit includes, as sensors configured to sense abnormalities, an acceleration sensor configured to sense an abnormality in a shock and a temperature sensor configured to sense an abnormality in a temperature, a communication time measurement unit configured to measure whether communication is completed within a predetermined time in order to sense an abnormality in communication, and a charging amount sensing unit configured to sense an abnormality in insufficient battery charging.

13. The system according to claim 1, wherein the examination information corresponding to the abnormality information contains information on a user of the radiation imaging apparatus that has caused the abnormality.

14. The system according to claim 1, further comprising a specifying unit configured to specify, based on the examination information corresponding to the abnormality information, a user of the radiation imaging apparatus that has caused the abnormality.

15. The system according to claim 14, wherein the specifying unit specifies, based on a history of authentication information input by a user, a last user who has used the radiation imaging apparatus.

16. The system according to claim 14, further comprising a measurement unit configured to measure an abnormality sensing count of the user who has caused the abnormality in the radiation imaging apparatus.

17. The system according to claim 15, wherein based on the input authentication information, the user is allowed to use the radiation imaging apparatus.

18. The system according to claim 14, wherein the abnormality information contains information indicating an abnormality level, and the examination information corresponding to the abnormality information contains information on the user of the radiation imaging apparatus that has caused the abnormality, and
the specifying unit specifies, based on the information indicating the abnormality level, a user of the radiation imaging apparatus that has caused a largest abnormality out of abnormalities occurred.

19. A control apparatus that controls a radiation imaging apparatus which detects radiation transmitted through an object and obtains a radiation image, the apparatus comprising:
an obtaining unit configured to obtain, from examination information stored in the control apparatus, examination information which corresponds to abnormality information indicating an abnormality occurring in the radiation imaging apparatus.

20. An information processing method in a radiation imaging apparatus which detects radiation transmitted through an object and obtains a radiation image, the method comprising:
obtaining abnormality information indicating an abnormality occurring in the radiation imaging apparatus; and
obtaining, from examination information stored in a storage unit, examination information corresponding to the abnormality information.

21. A storage medium storing a program for causing a computer to perform respective steps of an information processing method in a radiation imaging apparatus which detects radiation transmitted through an object and obtains a radiation image,
wherein the information processing method includes
obtaining abnormality information indicating an abnormality occurring in the radiation imaging apparatus, and
obtaining, from examination information stored in a storage unit, examination information corresponding to the abnormality information.
